(19) United States
(12) Patent Application Publication
(10) Pub. No.: US 2009/0041190 A1
(43) Pub. Date: Feb. 12, 2009

(54) Apparatus for Positioning Standing Persons in Order to Take X-Ray Pictures

(75) Inventor: Ruedi G. Laupper, Hitzkirch (CH)

Correspondence Address: THE NATH LAW GROUP, 112 South West Street, Alexandria, VA, 22314, US

(73) Assignee: SWISSRAY INTERNATIONAL INC., ELIZABETH, NJ (US)

(21) Appl. No.: 12/085,309

(22) PCT Filed: Nov. 24, 2005
(86) PCT No.: PCT/EP2005/056188

§ 371 Date: May 21, 2008

(51) Int. Cl. H05G 1/42; A61B 6/04

(52) U.S. Cl. 378/97; 5/601; 378/209

(57) ABSTRACT

An apparatus for positioning standing persons to produce X-ray shots comprises a movable frame on which a vertically displaceable standing area is arranged. The standing area has a holding apparatus connected to it which the person can hold onto. To produce optimum positioning in terms of weight too, the standing area has weighing apparatus arranged on it for determining the weight of the person, preferably using two independent weighing sensors for each foot of the person. A description is also given of an apparatus and a method for producing a digital X-ray image, where exposure parameters are calculated on the basis of person-related data and then a plurality of shots are taken sequentially and are subsequently used to compile an overall image.

The invention relates to an apparatus for positioning standing persons in order to take X-ray pictures in accordance with the preamble of claim 1. Such apparatuses serve the purpose of optimally positioning a patient, chiefly when applying special X-ray techniques.

Thus, for example, in the field of orthopedics it is necessary to take pictures of the feet under normal body loading. It is evidently necessary to this end that the patient must stand over the receiver arrangement or over the Bucky. In the case of the so-called long leg recording technique, a number of X-ray pictures are taken sequentially in a vertical position from the ankle up to the hip joint. It is also particularly important here that the patient be optimally positioned so that the X-ray pictures can be evaluated.

Various movable apparatuses are already known for positioning a patient in a vertical position. DE 100 56 798 describes a generic comparable apparatus in the case of which the standing surface can be displaced vertically on a column arrangement. Holders for X-ray film cassettes are arranged at or on the standing surface. A disadvantage of this apparatus consists in that no information of any sort regarding weight is available—in particular no information relating to the distribution of the body weight on the two feet.

It is therefore an object of the invention to provide an apparatus of the type mentioned at the beginning that enables a patient to be positioned exactly in terms of loading, as well, by taking account of weight information. Finally, it is intended that the apparatus also be suitable, in particular, for the modern digital X-ray technique, and, where possible, that in addition to the body weight further parameters of a person which are essential for calculating radiation dose can be detected. Of course, the apparatus is also intended to offer the patient advantages in terms of user friendliness.

This object is achieved according to the invention with the aid of an apparatus that has the features in claim 1. Owing to the arrangement of a weighing apparatus at the standing surface for determining the weight of the person, which preferably has a number of weighing sensors, the patient can be optimally placed and/or it is possible to correct faulty body postures. Appropriate weighing sensors that are capable of converting a pressure loading directly into an electrical signal are known to the expert and are therefore not described in more detail. The weighing apparatus is intended in any case also to be capable of detecting the total weight of the patient, because this is important for calculating the exposure parameters at the X-ray machine. The standing surface can be held in a frame, at least one weighing sensor, but preferably a number of weighing sensors, being arranged in the edge region of the frame. The weighing sensors can in this case be arranged in such a way that the weight distribution on the two feet can also be established on the same continuous standing surface. Alternatively, the standing surface could also be subdivided into separate partial standing surfaces for each foot, each partial standing surface being provided with separate weighing sensors.

Further advantages can be achieved when arranged above the standing surface is a sensor, preferably an ultrasonic sensor for measuring the body size of the person standing on the standing surface. Together with the weight of the patient, the body size also plays an important role in the differentiation of the exposure parameters. This prevents the occurrence of faulty exposures owing to defective estimates. The sensor can be arranged to be capable of displacement in depth such that it can be aligned exactly with the person respectively standing on the standing surface.

Further advantages can be attained when a sensor for determining the body fat content of the person is arranged on the apparatus.

Such sensors, which either measure the electrical resistance of the person or which operate with the aid of optical sensors, are likewise already known to the specialist and frequently used by him. Again, the measurement of body fat permits the radiologist to determine the radiation dose exactly.

It is particularly advantageous to arrange on the apparatus a transmitter for the wireless transmission of at least the body weight and, preferably, further person-related data determined by sensors to an X-ray machine. These data can in this case flow directly into a processing program that controls the X-ray operation. Clearly, errors such as could arise, for example, when inputting at a data console are thus avoided.

It is possible to arrange over the standing surface a frame structure that is designed at least partially as holding apparatus that serves a person standing on the standing surface for holding fast. The frame structure is designed in this case such that it can fulfill its purpose for persons of different body size.

Furthermore, fastening means for fixing the patient can be arranged on the apparatus. It can thereby be ensured that in the case of a number of X-ray pictures that must be subsequently combined a patient does not have his position displaced.

A display device for displaying person-related data can be arranged on the frame structure. This can be, for example, a liquid crystal display. However, the display device can also comprise a row of light emitting diodes that displays the symmetrical foot loading of the patient on the standing surface to the radiologist. The display device can be integrated directly into the apparatus itself, for example into the frame structure. However, it would also be conceivable in specific cases to design the display device as a separate unit that receives the data from the appropriate sensors via a cable or in a wireless fashion.

The frame structure advantageously has two parallel straight and vertical supports that are connected at their lower end to one horizontal supporting arm each, the standing surface being arranged between the two supporting arms. The holding apparatus can be provided in this case as two parallel, approximately convexly curved supports that are connected at their lower end to the horizontal supporting arms. The convex curvature of the supports for the holding apparatus is ergonomically advantageous, on the one hand. On the other hand, the patient can be well observed from the side.

A rear wall made from a material transparent to X-radiation can be arranged between the straight supports. A person standing on the standing surface can lean against this rear wall and straighten up. The rear wall does not appear in the X-ray image. Plexiglass can be involved here, or another material transparent to radiation. It would also be conceivable for a matrix pattern absorbing X-radiation to be arranged in the rear wall. This matrix pattern facilitates determinations of size, measurement operations and the combination of a number of images. The rear wall can also be designed as entry door for stepping onto the standing surface, something which substantially improves the comfort of operation.

Transporting the apparatus is facilitated by virtue of the fact that at least one upper end section of the frame structure is designed such that it can be lowered or swiveled away in order to reduce the overall height. Normal doors can be passed through without a problem in this way.

The comfort of operation for the patient can be further improved by virtue of the fact that an extendable stepladder or a staircase is arranged at the standing surface. The patient can step onto the standing surface in its already raised state without a problem in this way.

With reference to the structural design, the frame advantageously has two vertical guide cheeks between which the standing surface is guided, the guide cheeks being interconnected with the aid of a low-lying strut arranged near the floor. This low-lying strut facilitates pushing the Bucky under the raised standing surface. Nevertheless, the standing surface can be lowered in practice to floor level, and this facilitates the patient's entry. The vertical guide cheeks are connected at the lower end to one horizontal chassis strut each, thus producing an approximately L-shaped configuration seen from the side.

Raising the standing surface can be performed via a pumping mechanism or hydraulic apparatus that can be operated by a foot pedal. Such apparatuses are also known, for example, for operating tables and the like. They operate independently of current and reliably, and can also be used to move relatively high loads. However, it would also, of course, be conceivable to actuate the height adjustment with the aid of a hydraulic pump or by means of an electric motor.

The hydraulic device can have a pressure fluid cylinder extending approximately parallel to one of the chassis struts. The linear relative movement of the pressure fluid cylinder can in this case be transmitted via a chain gear to a sprocket shaft that extends over both guide cheeks, the sprocket shaft driving in each guide cheek a lifting chain with the aid of which the standing surface is raised. This design of the mechanism has the advantage that the force for the lifting movement is distributed uniformly over the two guide cheeks. Moreover, an ideal transmission ratio can thereby be produced relatively easily.

The invention also relates to a method for generating a digital X-ray image, in particular by using an apparatus described above, in which case in order to control the radiation dose exposure, parameters are firstly determined on the basis of person-related data of the person to be X-rayed and/or on the basis of the body part to be X-rayed. Subsequently, a number of digital X-ray pictures of the body part to be X-rayed are taken sequentially, with the recording field for each picture preferably being linearly displaced, and the image regions of successive pictures overlapping at least partially. Each individual picture is displayed on a display device and, if appropriate, processed by image processing measures such as contrast alteration or the like, for example. Subsequently, the individual pictures are combined to form an overall image and displayed and/or stored. The image processing can also be performed on the overall image. The combination of the individual images is performed digitally in a computer that is capable of detecting and combining over-lapping image regions.

This X-ray method is suitable, in particular, for recording extremities or for recording the spinal column. Conjoining individually produced X-ray images results in substantially better pictures compared with the conventional individual image technique. The process sequences can largely be automated in this case, and so operating errors can largely be eliminated.

The person-related data, in particular the body weight and the body size, can be determined by sensors before taking the X-ray pictures and can be fed to a radiation dose computer. As a result of picking up these parameters directly with the aid of sensors, it is always current values that are produced and defective estimated values are excluded. However, the person-related data, in particular the age of the person to be X-rayed, can also be retrieved from a data memory and fed to the radiation dose computer. It is also possible to conceive a combined feeding of data into the radiation dose computer via sensors, from a memory or by direct inputs at an input console.

The body mass index of the person to be X-rayed is advantageously determined from the person-related data, it being possible to correlate this index directly with the exposure parameters.

The number of individual images can be predetermined by a sequence selection device on the basis of the dimension of the body part to be X-rayed, the data of the sequence selection device being used to drive an actuator that displaces the X-ray imaging apparatus after each individual picture. Thus, for example, it will be necessary to prepare more individual images for recording the spinal column of a large person than for the arm of a child.

It is preferred for both the individual images and the combined overall image to be displayed on a common display screen. This renders it easier for the radiologist to judge the quality of the pictures taken and/or the quality of the combination of images.

An apparatus for carrying out the described method advantageously has the features in claim 25.

Further individual features and advantages of the invention emerge from the following description of an exemplary embodiment and from the drawings, in which:

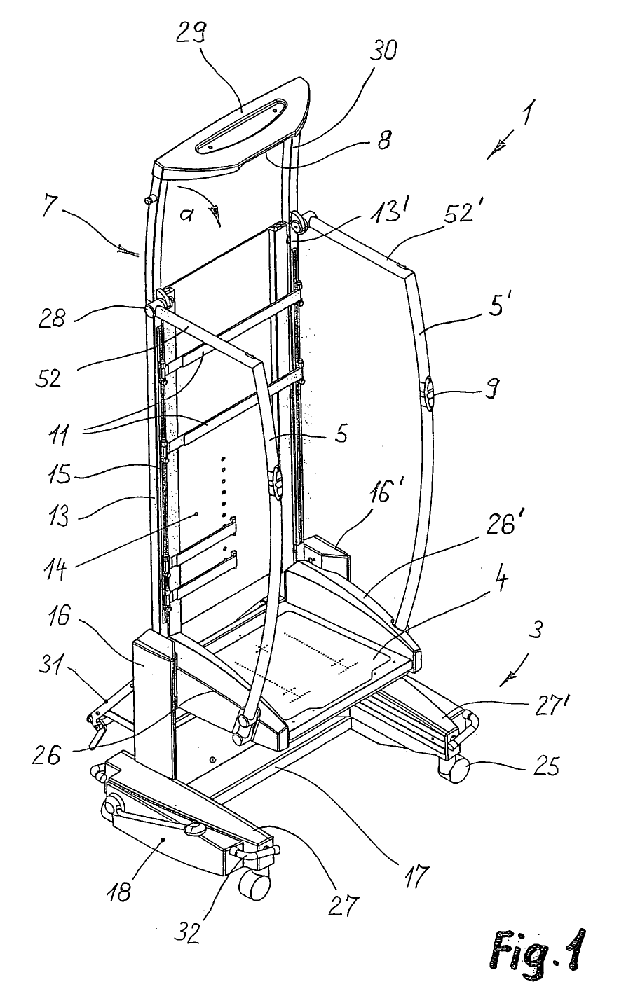

FIG. 1 shows a perspective illustration of an inventive apparatus, from the front,

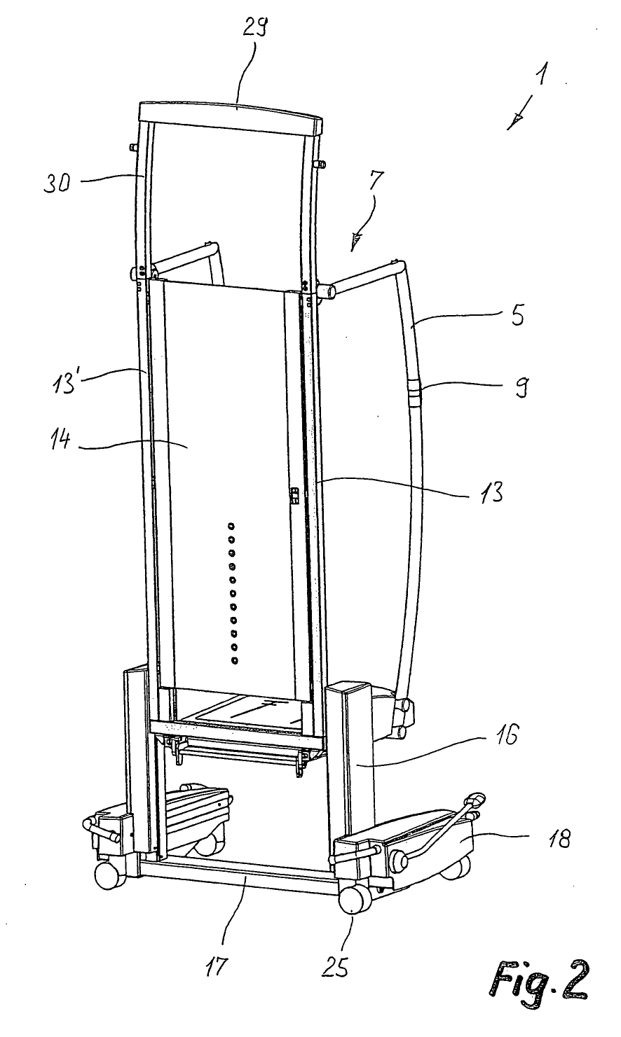

FIG. 2 shows the apparatus in accordance with FIG. 1, from the rear,

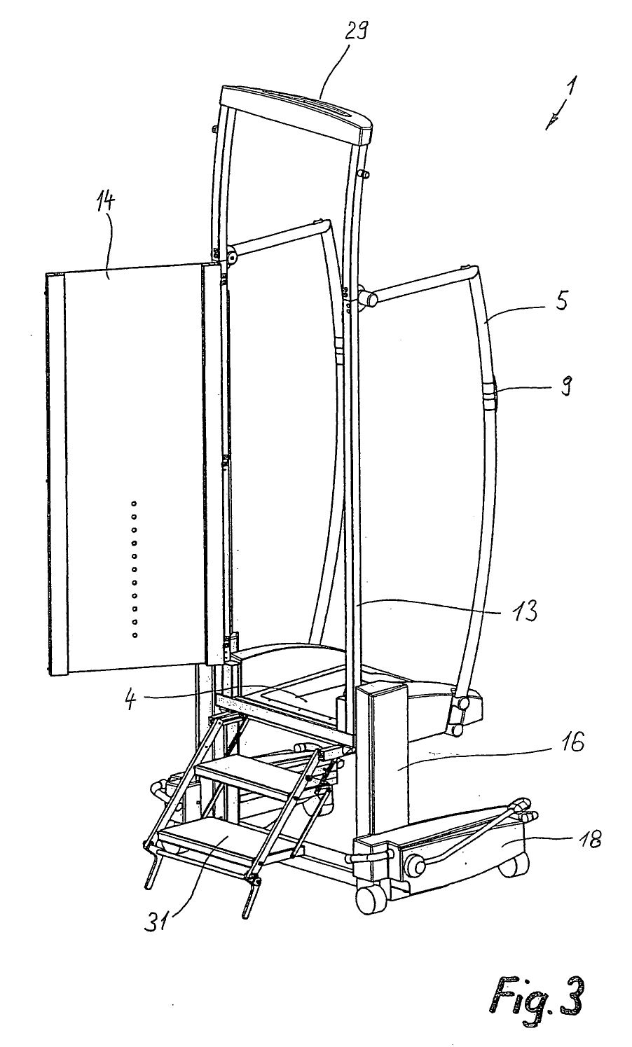

FIG. 3 shows the apparatus in accordance with FIG. 2, with open entry door and with staircase extended,

FIG. 4 shows a plan view of the standing surface of the apparatus in accordance with FIG. 1,

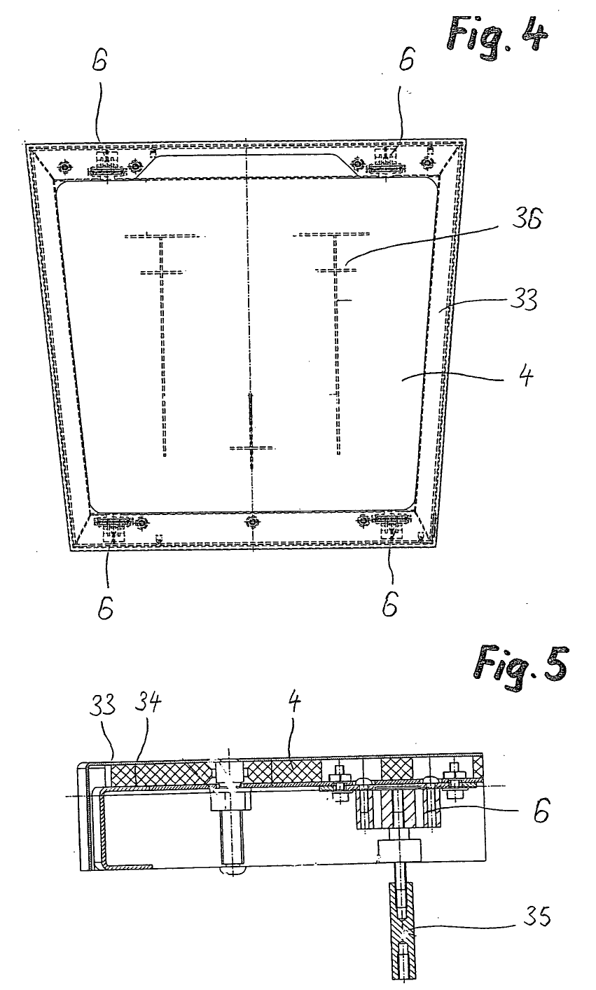

FIG. 5 shows an enlarged detailed cross section through a weighing sensor at the standing surface in accordance with FIG. 4,

FIG. 6 shows a perspective illustration of the pump mechanism in the removed state,

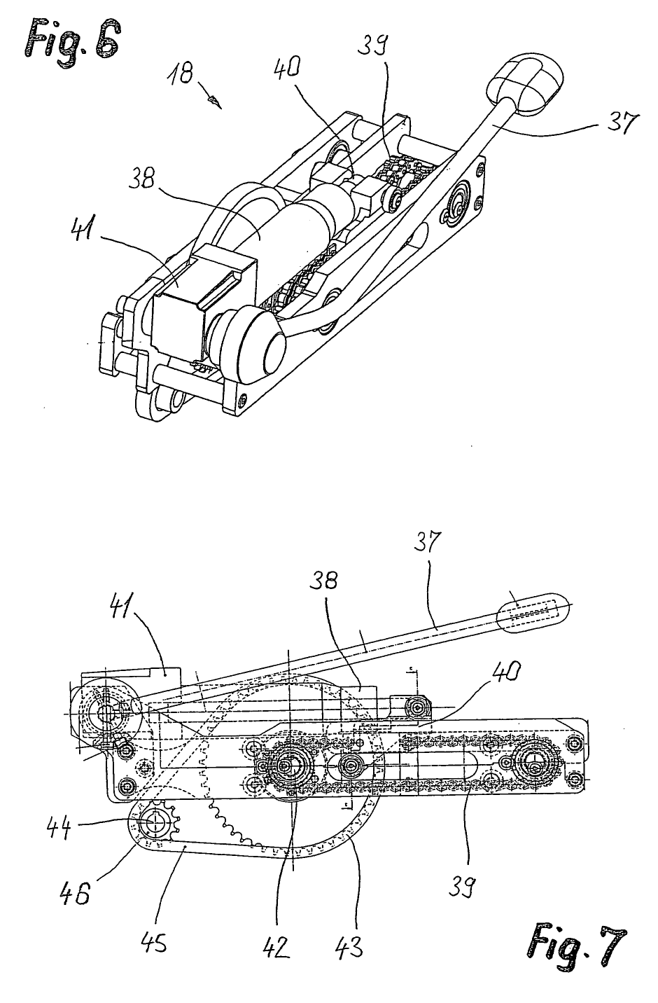

FIG. 7 shows a lateral illustration of the pump mechanism in accordance with FIG. 6,

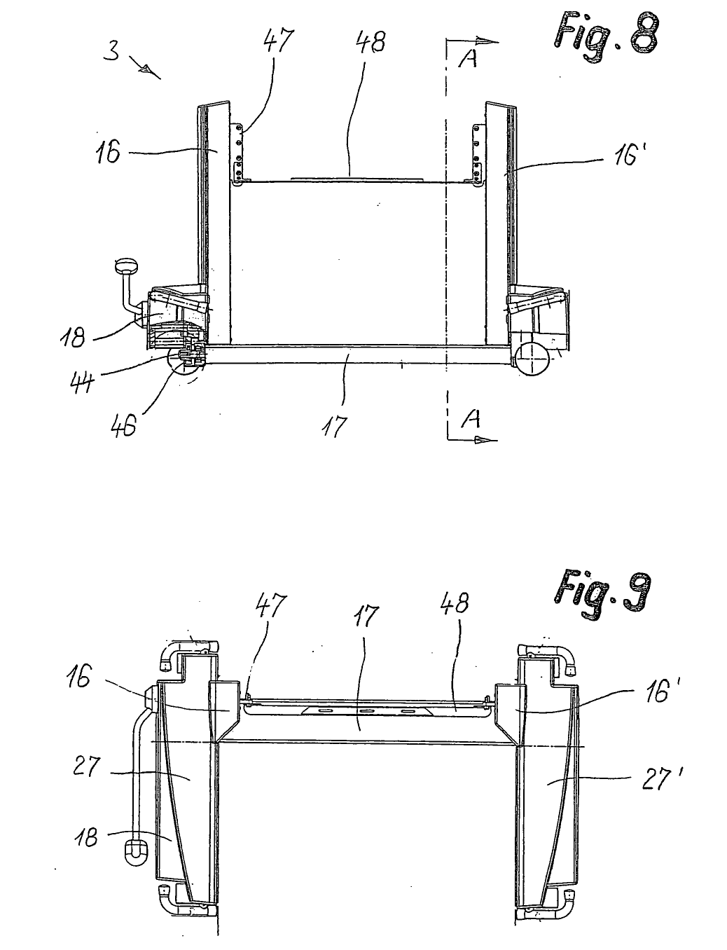

FIG. 8 shows a view from the front of the frame of the apparatus in accordance with FIG. 1,

FIG. 9 shows a plan view of the frame in accordance with FIG. 8,

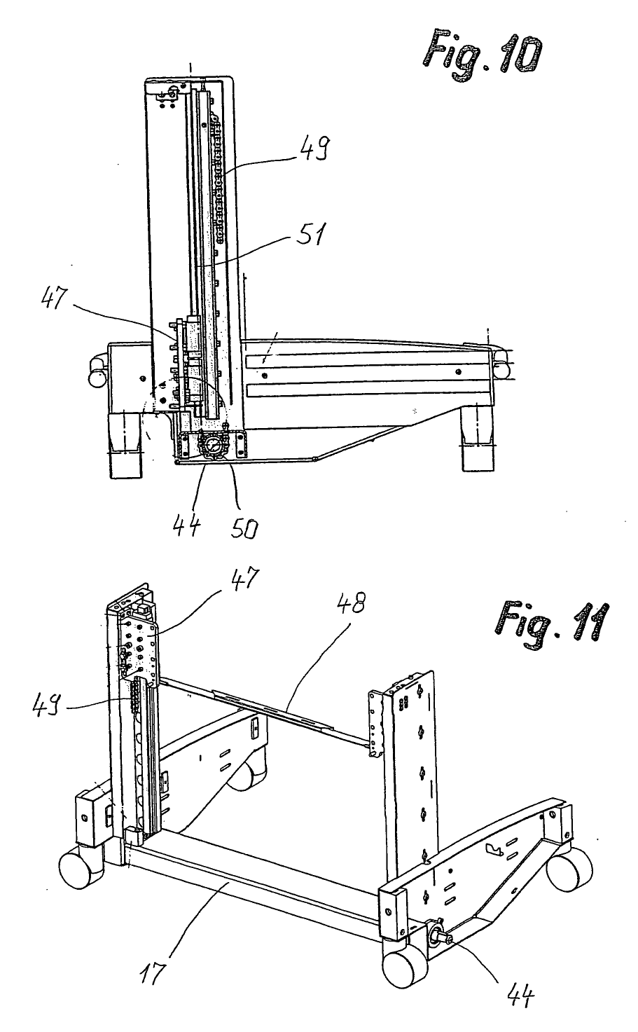

FIG. 10 shows a cross section through the plane A-A at the frame in accordance with FIG. 8,

FIG. 11 shows a perspective illustration of the frame in accordance with FIG. 8, from the rear with covering removed,

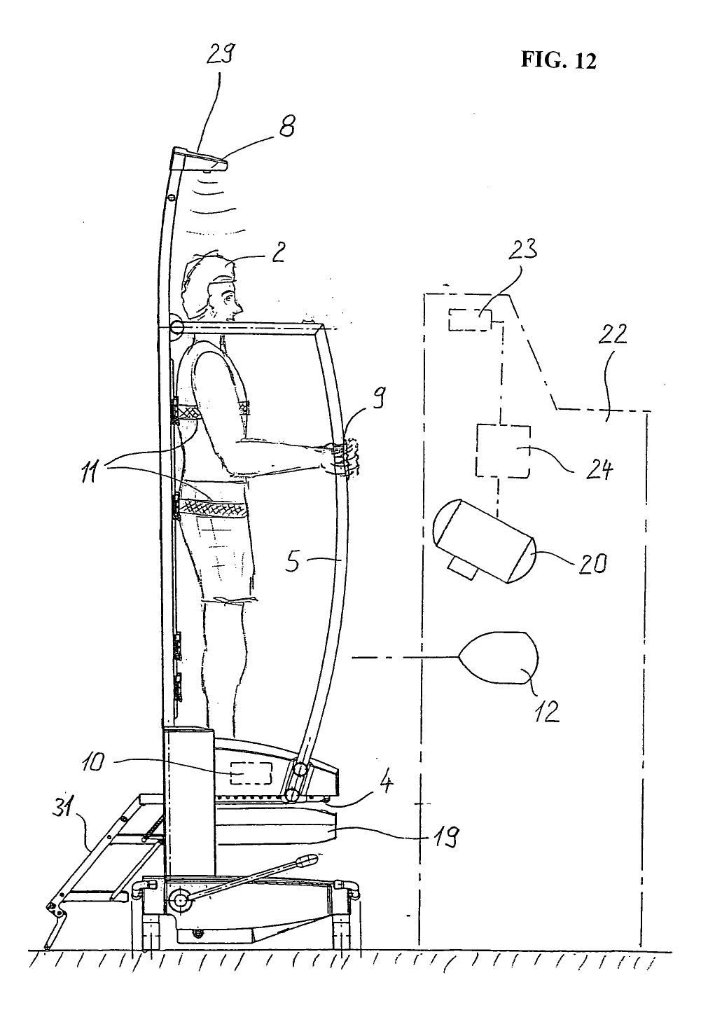

FIG. 12 shows a side view of the apparatus in accordance with FIG. 3, with a person positioned therein,

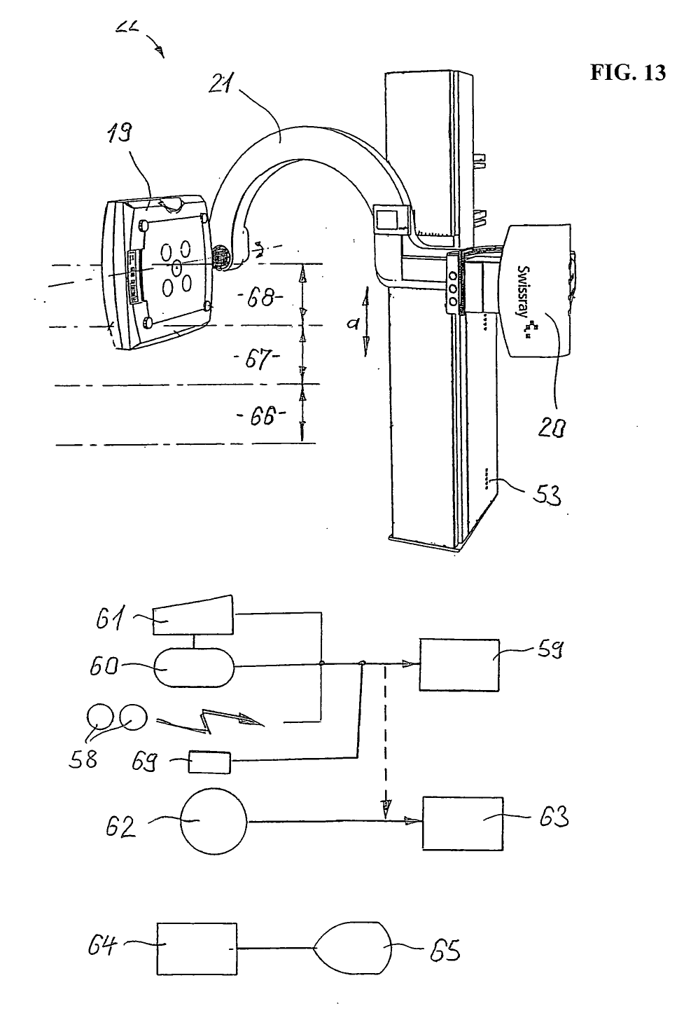

FIG. 13 shows a perspective illustration of an X-ray machine, with schematic completion of the components for image processing, and

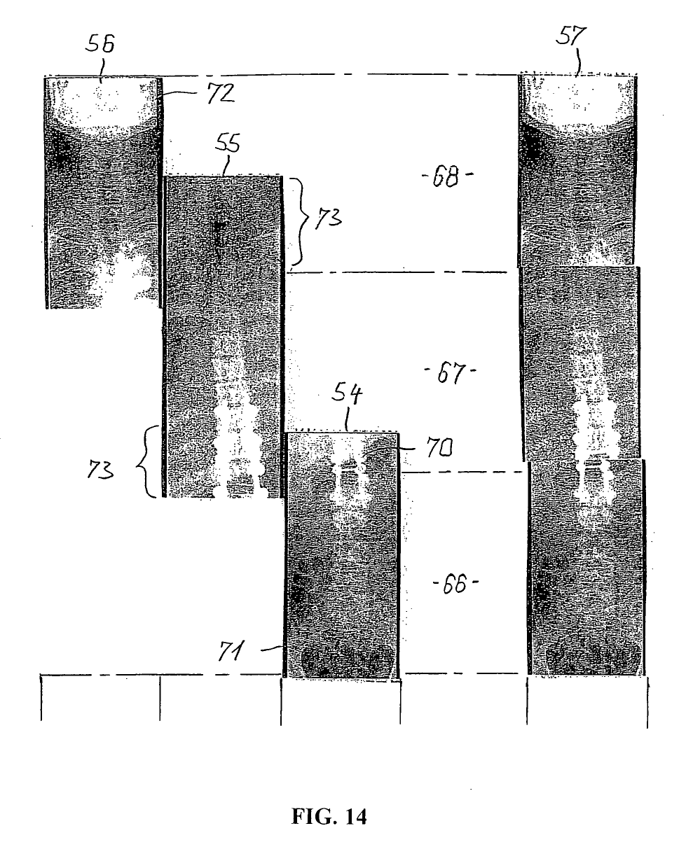

FIG. 14 shows the display of an X-ray image comprising three separate individual pictures.

As illustrated in FIG. 1, the apparatus denoted overall by 1 comprises a frame 3 that can be moved by means of steering rollers 25. Here, the frame essentially comprises two parallel side cheeks 16, 16′ of vertical alignment. These are connected to one chassis strut 27, 27′ each, the struts being of horizontal alignment such that an L-shaped arrangement results on each side of the frame. The two side cheeks 16, 16′ are interconnected via a low-lying transverse strut 17 arranged directly near the floor. Each steering roller 25 is assigned a roller brake that can be actuated via a brake lever 32.

Guided in each guide cheek 16, 16′ is a supporting arm 26, 26′ that extends approximately parallel to the chassis strut 27, 27′. Arranged between two supporting arms 26 is a standing surface 4 that must, of course, consist of a material transparent to radiation. Provided for the vertical height adjustment of the standing surface 4 is a pump mechanism 18 that can be actuated via a foot pedal. The design of the standing surface 4 and of the pump mechanism 18 will be described in yet more detail below. The same applies to the design of the frame 3.

A construction in the form of a frame structure 7 is arranged over the standing surface 4. Said structure comprises two parallel vertical and straight supports 13, 13′ and two convexly curved supports 5, 5′ that simultaneously form a holding apparatus for the patient on which he can hold fast. A rear wall 14 made from material transparent to radiation is arranged between the two vertical supports 13, 13′. The rear wall is in this case designed as a door that can be swiveled open, as may be seen, in particular, from FIG. 3. Moreover, fastening means 11 in the form of belts for fixing a patient are arranged in the region of the rear wall and the door 14. Fastened to this end on the vertical supports 13, 13′ are rails 15 in which the fastening means 11 are held such that they can be displaced in height. As illustrated, it is possible in this case for individual belts to extend over the entire width of the frame, while other ones can be fixed in the middle of the rear wall 14.

An upper section 30 of the frame structure 7 is swivelably arranged on hinges 38 such that it can be folded down in the direction of the arrow a. In the folded down state, this section rests on the horizontal bars 52, 52′, which merge into the holding apparatus 5, 5′.

Arranged in the uppermost region of the swiveling section 30 is a roof strut 29 on whose underside there is fastened an ultrasonic sensor 8 for measuring the size of the patient.

A folding staircase 31 (FIG. 3) is provided for stepping onto the standing surface 4. As may be seen from the figure, the folding staircase can be completely retracted without thereby impairing the function of the apparatus.

Arranged on the holding apparatus 5, 5′ are body fat sensors 9 that the patient must touch by holding fast. The body fat content can thus be determined via a measurement of electrical resistance, for example.

The design of the standing surface is explained below in more detail with the aid of FIGS. 4 and 5. The actual standing surface 4, comprising a plexiglass plate, for example, is retained in a frame structure that comprises an outer frame 33 and an inner frame 34. Arranged approximately in the corner region of this frame structure is one weighing sensor 6 each that is mounted on a support bolt 35. Arranged in the standing surface 4 are foot marks 36 that indicate the correct foot position to the patient. Depending on weight distribution, the four weighing sensors can determine whether the weight distribution is symmetrical or not. At the same time, the weighing sensors also determine the total weight of the patient standing on the standing surface 4.

Details of the pump mechanism for raising the standing surface 4 are to be seen from FIGS. 6 and 7. The pump 41 is actuated in this case via a pump lever 37. Each swiveling movement of the lever effects a linear displacement of the piston in the pressure fluid cylinder 38. This linear movement is transmitted with the aid of an engagement member 40 to a push chain 39 that is mounted below the pressure fluid cylinder 38. The push chain 39 meshes with a drive sprocket 42 that drives a transmission sprocket 43. The rotary movement is transmitted via a drive chain 45 to the sprocket 46 and to a sprocket shaft 44. The sprocket shaft 44 is arranged inside the low-lying strut 17 in the frame 3, as is to be seen, in particular, from FIGS. 8 to 11.

A lifting sprocket 50 is arranged on the sprocket shaft 44 in the region of each of the two guide cheeks 16, 16′. Each lifting sprocket 50 meshes with a lifting chain 49 (illustrated only partially in FIGS. 10 and 11). One fastening cheek 47 each is fixed on each lifting chain, these two fastening cheeks being interconnected by means of a connecting bar 48. The fastening cheeks 47 are guided on a vertical rectilinear guide 51. This transmission clearly effects a uniform force distribution during raising of the two supporting arms 26, 26′ (FIG. 1), which are not illustrated in FIGS. 8 to 11. FIG. 10 shows the fastening cheeks 47 in the lowermost position, and FIG. 11 shows these in the uppermost one.

FIG. 12 shows in terms of position the situation of the apparatus in accordance with FIGS. 1 and 2, a person 2 being positioned on the standing surface 4. The person is holding onto the holding apparatus 5 in the region of the body fat sensors 9 such that the body fat content is automatically determined. At the same time, the ultrasonic sensor 8 measures the body size of the person, whose weight is determined at the weighing sensors in the standing surface 4. The data thus established are transmitted in wireless fashion to a receiver 23 in the X-ray machine 22 via a transmitter 10. A radiation source 20 is directed obliquely from above onto the standing surface 4 or onto the feet of the person 2 such that the beams impinge on the Bucky 19 below the standing surface 4. Taking account, if appropriate, of further person-related data, such as age, a computer 24 determines the correct exposure parameters and the correct radiation dose. The data determined can be displayed, possibly also in analog or digital fashion, on a display device 12.

The X-ray machine 22 can be a machine known per se, for example for generating digital X-ray images. The Bucky 19 can in this case be swiveled on a C-arm into various positions. The design of the frame permits the Bucky to be lowered between the two chassis struts 27 virtually as far as onto the floor. As is to be seen, the Bucky 19 can also be positioned along the rear wall 14 or else laterally in various planes. This technique can be used to make sequential X-ray pictures of various body sections that can, in turn, be combined to form an overall image with the aid of digital X-ray technology.

FIG. 13 shows the overall view of an X-ray machine 22 with a C-arm 21 on which an X-ray receiver, for example a flat panel Bucky 19, is arranged at one end, and an X-ray source 20 is arranged at the other end. Via a drive (not illustrated here), the C-arm 21 can be adjusted in height on a frame 53 in the direction of the arrow a. It is to be seen that the patient frame illustrated in FIG. 12 can be placed between the Bucky 19 and the radiation source 20 in such a way that the Bucky 19 is arranged behind the frame rear wall 14 (FIG. 2) and can be moved in a plane parallel fashion relative to this into various height positions. Here, the patient stands with his/her back to the Bucky 19. A lateral placement of the patient would also be possible, however.

The control for the X-ray machine 22 has a radiation dose computer 59 that sets the correct radiation dose on the basis of specific exposure parameters at the radiation source 20. These exposure parameters are a function of person-related data of the patient such as, for example, weight, body size, body fat content or age. The person-related data can either be input at an input console 61 or, at best, can also be read in via a patient code.

The data can also already be stored in a data memory 60 and be retrieved therefrom. As described above, at least some of the person-related data can be determined directly at the patient via sensors 58 and, for example, be fed in wireless fashion to the radiation dose computer 59. The radiation dose is ultimately also a function of the body part to be X-rayed, the radiologist being able to select the latter via a selection switch 69. The selection switch can, for example, be a touch sensitive display screen on which a skeleton is displayed.

Depending on the size of the body part to be X-rayed, it is necessary to take a number of overlapping X-ray pictures one after the other. The number of pictures is determined by a sequence selection device 62 that, for its part, drives an actuator 63. This actuator ensures that the C-arm 21 moves successively to a first imaging position 66, a second imaging position 67 and a third imaging position 68. The sequence selection device 62 can be configured such that it itself determines the number of image sequences as a function of the person-related data and/or as a function of the position of the selection switch 69. However, it is also possible for the radiologist to intervene manually in the sequence selection device 62.

The control of the X-ray machine then further has an image composition device 64 for storing and combining the X-ray images, produced in each imaging position, with the aid of a display device 65 for displaying the digitally stored and/or combined X-ray images.

The combination of individual X-ray images is illustrated in simplified fashion in FIG. 14. A picture of the entire spinal column from the pelvic region 71 up to the skull region 72 is taken for a patient having an implant 70 on the spinal column. To this end, a first digital X-ray image 54 is taken in the first imaging position 66. Following thereafter in the second imaging position 67 is the taking of a second X-ray image 55 and, finally, taking of the third X-ray picture 56 in the third imaging position 68. As illustrated, they result in the case of the successive pictures in overlap regions 73 in which a part of the neighbouring adjacent picture is still visible in each case.

The individual images 54, 55 and 56 can be combined to form an overall image 57 with the aid of special software in the image composition device 64. Here, the overlap regions are eliminated such that a virtually seamless overall image results. The quality of the X-ray image with reference to resolution, contrast action etc. can be influenced by special measures as early as in the individual images, or in the overall image. The inventive X-ray method with digital image composition can be implemented on all X-ray machines with digital image conversion, specifically including on lying patients, for example. The method can, however, be applied with particular advantage in combination with the frame described with the aid of FIGS. 1 to 12, it being possible for specific person-related data to be determined directly via sensors.

The following process takes place when an X-ray image illustrated in accordance with FIG. 14, for example, is taken: In accordance with FIG. 12, for example, the radiologist places the patient on the positioning device and firstly retrieves the person-related data by actuating a switch. The sensors determine, for example, a body size of 162 cm and a body weight of 59 kg, from which a body mass index of 22 is calculated.

Subsequently, the selection switch is actuated in order to select the body part to be X-rayed, that is to say the “spinal column”, for example. The sequence selection device now makes a proposal for the number of image sequences, that is to say three individual images in the present case. The proposal can either be confirmed by pressing a button, or it can be changed by selecting another sequence.

The Bucky 19 now moves into the first imaging position 66, here, as well, it being possible to overcontrol for fine adjustment by virtue of the fact that the Bucky can still be displaced from the approached basic position into the final recording position. The computer uses the definitive start position to determine the second and the third imaging position and the path to be covered in this case. Before the activation of the radiation source, the radiologist is shown the radiation dose determined or proposed on the basis of the body mass index. Of course, it is also possible here to intervene manually in the control and to vary the radiation dose.

Subsequently, the first X-ray image 54 is taken by actuating an actuating button, and displayed to the radiologist on a display screen or on a partial surface of a display screen. If the partial picture is satisfactory, the next imaging position 67 can automatically be approached, and the operation is repeated for the second X-ray image and the third X-ray image.

After the last picture, a digital combination of the individual images to form an overall image is performed automatically, said overall image likewise being imaged on the display screen next to the individual images. The operator can now further process the image by digital measures, something which is, if appropriate, also already possible on the partial images. Subsequently, the overall image can be accepted by actuating a switch, whereupon said overall image is stored in digital form in the data memory and can again be retrieved, printed out or transmitted via data lines at any time.

1-25. (canceled)

26. An apparatus for positioning standing persons in order to take X-ray pictures, having a frame on which a vertically displaceable standing surface is arranged, wherein a weighing apparatus for determining the body weight of the person standing on the standing surface is arranged on the standing surface.

27. The apparatus as claimed in claim 26, wherein the standing surface is held in a frame, and in that at least one weighing sensor is arranged in the edge region of the frame.

28. The apparatus as claimed in claim 26, wherein a number of weighing sensors are arranged on the standing surface in such a way that, in addition to the body weight of the person, the weight distribution on the person's feet is also determinable.

29. The apparatus as claimed in claim 26, wherein arranged above the standing surface, a sensor is arranged for determining the body size of the person standing on the standing surface.

30. The apparatus as claimed in claim 26, wherein it has a sensor for determining the body fat content of the person standing on the standing surface.

31. The apparatus as claimed in claim 26, wherein it has a transmitter for the wireless transmission of at least the body weight to an X-ray machine.

32. The apparatus as claimed in claim 26, wherein it has a display device for displaying person-related data determined by sensors.

33. The apparatus as claimed in claim 26, wherein above the standing surface, a frame structure is arranged that is designed at least partially as holding apparatus that serves the person standing on the standing surface for holding fast.

34. The apparatus as claimed in claim 33, wherein fastening means for fixing the person standing on the standing surface are arranged on the frame structure.

35. The apparatus as claimed in claims 33, wherein the frame structure has two parallel vertical supports that are connected at their lower end to one horizontal supporting arm each, the standing surface being arranged between the two supporting arms.

36. The apparatus as claimed in claim 35, wherein a rear wall made from a material transparent to X-radiation is arranged between the two parallel vertical supports.

37. The apparatus as claimed in claim 36, wherein the rear wall is designed as entry door for stepping onto the standing surface.

38. The apparatus as claimed in claim 33, wherein at least one upper end section of the frame structure is designed such that it can be lowered or swiveled away in order to reduce the overall height.

39. The apparatus as claimed in claim 26, wherein an extendable stepladder is arranged at the standing surface.

40. The apparatus as claimed in claim 26, wherein the frame has two vertical guide cheeks that are connected at the lower end to one horizontal chassis strut each, the standing surface being held between the guide cheeks and/or between the chassis struts.

41. The apparatus as claimed in claim 40, wherein the guide cheeks are interconnected with the aid of a low-lying strut arranged near the floor.

42. The apparatus as claimed in claim 40, wherein for the purpose of raising the standing surface, a hydraulic drive device is arranged on the frame that can be operated by a foot pedal and has a pressure fluid cylinder extending approximately parallel to one of the chassis struts.

43. The apparatus as claimed in claim 42, wherein the linear relative movement of the pressure fluid cylinder can be transmitted via a chain gear to a sprocket shaft that extends over both guide cheeks, and wherein in each guide cheek the sprocket shaft drives a lifting chain for raising the standing surface.

44. A method for generating a digital X-ray image, wherein in order to control the radiation dose exposure parameters are firstly determined on the basis of person-related data of the person to be X-rayed, and/or on the basis of the body part to be X-rayed, wherein subsequently a number of digital X-ray pictures of the body part to be X-rayed are taken sequentially, with the recording field for each picture preferably being linearly displaced, and the image regions of successive pictures overlapping at least partially, in that each picture is displayed on a display device and, if appropriate, processed by image processing measures, and in that the individual pictures are ultimately combined to form an overall image and displayed and/or stored.

45. The method as claimed in claim 44, wherein the person-related data are determined by sensors before taking the X-ray pictures and are fed to a radiation dose computer.

46. The method as claimed in claim 44, wherein the person related data are retrieved from a data memory and fed to a radiation dose computer.

47. The method as claimed in claim 44, wherein the body mass index (BMI) is determined from the person related data, and in that the exposure parameters are correlated therefrom.

48. The method as claimed in claim 44, wherein the number of individual images is predetermined by a sequence selection device on the basis of the dimension of the body part to be X-rayed, and in that the data of the sequence selection device are used to drive an actuator that displaces the X-ray imaging apparatus after each individual picture.

49. The method as claimed in claim 44, wherein both the individual images and the combined overall image are displayed on a common display screen.

50. An image generating apparatus for generating a digital X-ray image with the aid of means for determining the exposure parameters in order to control the radiation dose on the basis of person related data of the person to be X-rayed and/or on the basis of the body part to be X-rayed, having an X-ray sender/receiver unit, characterized by an image storage device for storing individual X-ray images, an actuator for preferably linear displacement of at least the X-ray receiver after each X-ray picture, in such a way that the image regions of successive pictures can overlap at least partially, an image composition device for combining the overlapping individual images to form an overall image, and by a display device for displaying both the individual images after each picture and the combined overall image.

51. Image generating apparatus according to claim 50, wherein the image generating apparatus comprises an apparatus for positioning standing persons in order to take X-ray pictures, having a frame on which a vertically displaceable standing surface is arranged, wherein a weighing apparatus for determining the body weight of the person standing on the standing surface is arranged on the standing surface.
